(19) United States
(12) Patent Application Publication
(10) Pub. No.: US 2005/0123946 A1
(43) Pub. Date: Jun. 9, 2005

(54) Methods for specific rapid detection of pathogenic food-relevant bacteria

(76) Inventors: Jiri Snaidr, Grossinzemoos (DE); Claudia Beimfohr, Munich (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/909,757

(22) Filed: Aug. 2, 2004

Related U.S. Application Data
(63) Continuation-in-part of application No. PCT/EP03/01092, filed on Feb. 4, 2003.

(30) Foreign Application Priority Data
Feb. 4, 2003 (DE) 102 04 447.3

(51) Int. Cl. C12Q 1/68; C07H 21/04

(52) U.S. Cl. 435/6; 536/24.1

(57) ABSTRACT

The invention relates to a method for the detection of pathogenic food-relevant bacteria, particularly to a method for the simultaneous specific detection of bacteria of the genus Listeria and the species Listeria monocytogenes by in situ-hybridization as well as to a method for the specific detection of bacteria of the species Staphylococcus aureus by in situ-hybridization as well as to a method for the specific detection of bacteria of the genus Campylobacter and the species C. coli and C. jejuni by in situ-hybridization as well as the corresponding oligonucleotide probes and kits, with which the inventive methods may be carried out.

### CROSS REFERENCE TO RELATED APPLICATION

The present application is a continuation-in-part of PCT application Serial No. PCT/EP03/01092, filed Feb. 4, 2003, entitled “METHODS FOR SPECIFIC RAPID DETECTION OF PATHOGENIC FOOD-RELEVANT BACTERIA,” the disclosure of which is incorporated herein by reference in its entirety; which claims priority from German Patent Application Serial No. 102 04 447.3, filed Feb. 4, 2002, the disclosure of which is incorporated herein by reference in its entirety.

### BACKGROUND OF THE INVENTION

1. Field of the Invention

The invention relates to a method for the detection of pathogenic food-relevant bacteria, particularly to a method for the simultaneous specific detection of bacteria of the genus Listeria and the species Listeria monocytogenes by in situ-hybridization as well as to a method for the specific detection of bacteria of the species Staphylococcus aureus by in situ-hybridization as well as to a method for the specific detection of bacteria of the genus Campylobacter and the species C. coli and C. jejuni by in situ-hybridization as well as the corresponding oligonucleotide probes and kits, with which the inventive methods may be carried out.

2. Description of the Related Art

Listeria are gram-positive short motile rods. Six species belong to the genus Listeria (L.): L. grayi, L. innocua, L. ivanovii, L. monocytogenes, L. seeligeri and L. welshimeri. The worldwide distribution of these ubiquitous bacteria extends to both aquatic areas as well as to soil and vegetation.

Listeria gain special medicinal importance because of an infectious disease known as Listeriosis caused in humans as well as in domestic and wild animals. In humans the listeriosis, which has a highly variable incubation period from a few days up to two months, is caused by the species L. monocytogenes, but in some diseases also L. ivanovii, L. seeligeri and L. welshimeri were detected. A listeria infection can manifest itself in severe diseases such as sepsis, meningitis or encephalitis. Especially in newborn infants, who can be infected via the placenta or during delivery, as well as in elderly people listeriosis carries a high health risk. The fatality rate in the case of newborn listeriosis is up to 50%. An infection prior to birth can result in the fetus being aborted. The occurrence of a listeriosis in elderly or otherwise immuno-compromised people can be fatal in up to 30% of those infected.

Transmission usually occurs from consuming contaminated foodstuffs. Especially milk products are a frequent source of infection. But also nearly all other foodstuffs are potential sources of listeria infections. Besides milk and various milk products such as cheese, butter or ice-cream, also other foodstuffs were identified as a source of listeriosis in the past. These include such diverse products as coleslaw, mussels, pork, chicken, fish, cornmeal, or rice salad. In many cases outbreaks of listeriosis caused by consumption of the mentioned foodstuffs have proved fatal.

Of particular importance in this connection is the fact that Listeria are able to multiply also at 4° C. (in milk even at −0.3° C.). This means that despite cool storage of foodstuffs, Listeria can multiply and accumulate in the foodstuffs. Even after cooking, roasting or smoking, Listeria may accumulate in the relevant foodstuffs as a consequence of insufficient treatment or a secondary contamination.

Therefore a continuous monitoring of foodstuffs for the occurrence of Listeria is an important part of both quality assurance in manufacturing companies as well as the daily routine in hygiene institutes.

The classic method for the detection of L. monocytogenes is very time-consuming. In this case, first an enrichment in a selective liquid medium, the so-called ½ Fraser bouillon is carried out at 30° C. for 24 hours. This is followed by a second enrichment step, now in Fraser bouillon, at 37° C. for 48 hours. Both enrichments are then plated on selective agar media (Oxford-Agar and PALCAM-Agar) and these are incubated at 30° C. or 37° C. for 24 hours to 48 hours. To confirm that the colonies grown in this way are Listeria or L. monocytogenes further sub-cultivations are made (on Trypton soya yeast agar or sheep's blood agar) for a period of at least 24 hours, to at most five days. The overall period of the classic detection method is therefore five to ten days.

Staphylococcus intoxications belong to the worldwide most prevalent diseases which are caused by bacteria and transmitted by foodstuffs. These are especially caused by strains of Staphylococcus (S.) aureus. S. aureus is a gram-positive, immotile, coagulase-positive bacterium and occurs on the skin, the mucosa of the nasopharynx, in stool, feces, abscesses and pustules. S. aureus is also widespread among the healthy population. S. aureus can be detected in the nasopharynx of half of all healthy people.

Food poisoning as a consequence of an infection with S. aureus is caused by enterotoxins produced by these bacteria in foodstuffs and is characterized by vomiting and diarrhea. Enterotoxin A has the strongest effect with an emetic dose of below 1 μg. Even 0.1-0.2 μm Enterotoxin lead to food poisoning. Toxin F also deserves special mention, which leads to a shock syndrome and is therefore also called “Toxic Shock Syndrome Toxin” (TSST-1). Characteristics of the shock syndrome caused by Toxin F are pulmonary edema, endothelial cell degenerations, renal failure and shock.

Transmission of S. aureus usually also occurs through the consumption of contaminated foodstuffs, the spectrum of potential sources of infection being quite wide. The following foodstuffs were involved among others in incidences of the disease: pre-cooked convenience foods containing meat, pies, ham, gammon, milk and milk products, egg-containing dishes, salads, creams, cake fillings, ice cream, and pasta.

Routine detection nowadays is performed mostly by cultivation and confirmation testing of suspect colonies, because enterotoxin detection is quite complicated to perform. For the detection by cultivation, the sample to be examined is first incubated for 48 hours on a suitable selective medium (e.g., Baird) at 37° C. If this first cultivation step was performed in liquid medium, a second one (again for 48 hours) follows on a solid medium (e.g., Baird-Parker). In the next step the suspect colonies are tested for the presence of coagulase. For this, two different methods are available. Usually, first the so-called “tube test for the presence of clotting factor” is performed, which takes about six to eight hours. If this test is negative, the result has to be confirmed by the so-called “tube test using rabbit plasma”. This test takes up to 24 hours. The overall period of the classic detection method is therefore between 54 hours and 5 days.

It has only been in the last 20-odd years that a previously underestimated germ has been playing a bigger role as food poisoner, namely Campylobacter. In contrast to, for instance, Salmonella, it rarely propagates in food, however, for an infection with this pathogen, even a few hundred bacterial cells are sufficient.

The genus Campylobacter (C.) comprises 20 species and sub-species. These bacteria, which have up to now been difficult to cultivate, are gram-negative, slender, curved to spirally curved rods, which require microaerophilic conditions for their growth.

Medically relevant are the species C. jejuni, C. coli and C. laris. They populate the small intestine and the colon and cause an acute gastroenteritis accompanied by the following symptoms: diarrhea, abdominal pain, fever, nausea, and vomiting. These symptoms are very difficult to distinguish from those of a gastric ulcer. A careful differential diagnosis is thus essential.

Presently the routine detection is performed via a multi-stage cultivation, beginning with an 18-hour enrichment in selective liquid medium (Campylobacter selective medium according to Preston), followed by two periods of 48 hours each on two different solid media (Karmali agar, followed by Columbia blood agar). These five-day cultivations are followed by the biochemical or serological identification.

As a logical consequence of the difficulties, especially the lengthiness, presented by the above-mentioned methods for the detection of Listeria, S. aureus and Campylobacter, detection methods on the basis of nucleic acids would be useful.

### SUMMARY OF THE INVENTION

Embodiments relate to isolated oligonucleotides for the simultaneous specific detection of bacteria of the genus Listeria and/or the species L. monocytogenes. For example the isolated oligonucleotides can include an oligonucleotide having a nucleic acid sequence selected from the group consisting of:

| | |
|---|---|
| SEQ ID NO. 1: | 5′-GGC TTG CAC CGG CAG TCA CT, |
| SEQ ID NO. 2: | 5′-CGG CTT ACA CCG GCA GTC ACT, |
| SEQ ID NO. 3: | 5′-CCC TTT GTA CTA TCC ATT GTA, |
| SEQ ID NO. 4: | 5′-CCC TTT GTA CCA TCC ATT GTA, |
| SEQ ID NO. 5: | 5′-CCC TTT GTA TTA TCC ATT GTA G, |
| and | |
| SEQ ID NO. 6: | 5′-CCC TTT GTA CTG TCC ATT GTA; |

an isolated oligonucleotide which is at least 60%, 80%, 90%, 92%, 94% or 96% identical to an oligonucleotide according to i) and which renders possible a specific hybridization with a nucleic acid sequence of a bacteria of the genus Listeria, preferably a bacteria of the species L. monocytogenes; an oligonucleotide which differs from the oligonucleotide according to i) and/or ii) in that it is extended by at least one nucleotide; and an oligonucleotide which hybridizes with a sequence complementary to an oligonucleotide according to i), ii) or iii) under stringent conditions.

Other embodiments relate to isolated oligonucleotides for the specific detection of bacteria of the species S. aureus. For example the isolated oligonucleotides can include an oligonucleotide having a nucleic acid sequence selected from the group consisting of:

an oligonucleotide having a nucleic acid sequence selected from the group consisting of:

| | |
|---|---|
| SEQ ID NO. 7: | 5′-GAA GCA AGC TTC TCG TCC G, |
| SEQ ID NO. 8: | 5′-GGA GCA AGC TCC TCG TCC G, |
| SEQ ID NO. 9: | 5′-GAA GCA AGC TTC TCG TCA TT, |
| SEQ ID NO. 10: | 5′-CTA ATG CAG CGC GGA TCC, |
| SEQ ID NO. 11: | 5′-CTA ATG CAC CGC GGA TCC, |
| SEQ ID NO. 12: | 5′-CTA ATG CGG CGC GGA TCC, and |
| SEQ ID NO. 13: | 5′-CTA ATG CAG CGC GGG TCC; |

an oligonucleotide which is at least 60%, 80%, 90%, 92%, 94% or 96% identical to an oligonucleotide according to i) and which renders possible a specific hybridization with a nucleic acid sequence of the species S. aureus; an oligonucleotide which differs from the oligonucleotide according to i) and ii) in that it is extended by at least one nucleotide; and an oligonucleotide which hybridizes with a sequence complementary to an oligonucleotide according to i), ii) or iii) under stringent conditions.

Still further embodiments relate to isolated oligonucleotides for the simultaneous specific detection of bacteria of the genus Campylobacter and the species C. coli and/or C. jejuni. For example the isolated oligonucleotides can include an oligonucleotide having a nucleic acid sequence selected from the group consisting of:

an oligonucleotide having a nucleic acid sequence selected from the group consisting of:

| | |
|---|---|
| 5′ CTG CCT CTC CCT CAC TCT AG, | SEQ ID NO. 16 |
| 5′ CTG CCT CTC CCT TAC TCT AG, | SEQ ID NO. 17 |
| 5′ CTG CCT CTC CCC TAC TCT AG, | SEQ ID NO. 18 |
| 5′ CTG CCT CTC CCC CAC TCT AG, | SEQ ID NO. 19 |
| 5′ CCT ACC TCT CCC ATA CTC TAG A, | SEQ ID NO. 20 |
| 5′ CCA TCC TCT CCC ATA CTC TAG C, | SEQ ID NO. 21 |
| 5′ CCT ACC TCT CCA GTA CTC TAG T, | SEQ ID NO. 22 |
| 5′ CCT GCC TCT CCC ACA CTC TAG A, | SEQ ID NO. 23 |
| 5′ CGC TCC GAA AAG TGT CAT CCT C, | SEQ ID NO. 24 |
| 5′ CTA AAT ACG TGG GTT GCG, | SEQ ID NO. 25 |
| 5′ CTA AAC ACG TGG GTT GCG, | SEQ ID NO. 26 |
| 5′ AGC AGA TCG CCT TCG CAA T, | SEQ ID NO. 27 |
| 5′ AGC AGA TCG CTT TCG CAA T, | SEQ ID NO. 28 |
| 5′ AGT AGA TCG CCT TCG CAA T, | SEQ ID NO. 29 |
| 5′ TCG AGT GAA ATC AAC TCC C, | SEQ ID NO. 30 |
| 5′ TCG GGT GAA ATC AAC TCC C, | SEQ ID NO. 31 |
| 5′ CGT AGC ATG GCT GAT CTA C, | SEQ ID NO. 32 |
| 5′ CGT AGC ATA GCT GAT CTA C, | SEQ ID NO. 33 |
| 5′ CGT AGC ATT GCT GAT CTA C, | SEQ ID NO. 34 |
| 5′ GCC CTG ACT AGC AGA GCA A, | SEQ ID NO. 35 |
| 5′ TTC TTG GTG ATC TCT ACG G, | SEQ ID NO. 36 |
| 5′ TTC CTG GTG ATC TCT ACG G, | SEQ ID NO. 37 |
| 5′ TTC TTG GTG ATA TCT ACG G, | SEQ ID NO. 38 |
| 5′ TTG AGT TCT AGC AGA TCG C, | SEQ ID NO. 39 |
| 5′ TTG AGT TCC AGC AGA TCG C, | SEQ ID NO. 40 |
| 5′ TTG AGT TCT AGC AGA TAG C, | SEQ ID NO. 41 |
| 5′ TTG AGT TCC AGC AGA TAG C, | SEQ ID NO. 42 |
| 5′ CGC GCC TTA GCG TCA GTT GAG, | SEQ ID NO. 43 |
| 5′ CAC GCC TTA GCG TCA GTT GAG, | SEQ ID NO. 44 |
| 5′ CGC GCC TTA GCG TCA GTT AAG, | SEQ ID NO. 45 |
| 5′ CAC GCA TTA GCG TCA GTT GAG, | SEQ ID NO. 46 |
| 5′ CGA GCA TTA GCG TCA GTT GAG, | SEQ ID NO. 47 |
| 5′ TAC ACT AGT TGT TGG GGT GG, and | SEQ ID NO. 48 |
| 5′ TTC GCG CCT CAG CGT CAG TTA CAG; | SEQ ID NO. 49 |

an oligonucleotide which is at least 60%, 80%, 90%, 92%, 94% or 96% identical to an oligonucleotide according to i) and which renders possible a specific hybridization with a nucleic acid sequence of bacteria of the genus Campylobacter, preferably a bacteria of the species C. coli and/or C. jejuni; an oligonucleotide which differs from an oligonucleotide according to i) and ii) in that it is extended by at least one nucleotide; and an oligonucleotide which hybridizes with a sequence complementary to an oligonucleotide according to i), ii) or iii) under stringent conditions.

Some embodiments relate to methods for the simultaneous specific detection of bacteria of the genus Listeria in a sample. The methods can include, for example, the steps of: cultivating pathogenic food-relevant bacteria contained in a sample; fixing the pathogenic food-relevant bacteria present in the sample; incubating the fixed bacteria with at least one oligonucleotide above and herein, in order to achieve hybridization; removing non-hybridized oligonucleotide; and detecting and visualizing pathogenic food-relevant bacterial cells of the genus Listeria, preferably L. monocytogenes, with the hybridized oligonucleotide. The methods can further include the step of quantifying the pathogenic food-relevant bacterial cells with the hybridized oligonucleotide. The sample can be, for example, a foodstuff sample. The detection can be performed, for example, by an optical microscope, an epifluorescence microscope, a chemoluminometer, a fluorometer, or a flow cytometer.

Other embodiments relate to methods for the specific detection of bacteria of the species S. aureus in a sample. The methods can include, for example, the steps of: cultivating pathogenic food-relevant bacteria contained in a sample; fixing the pathogenic food-relevant bacteria present in the sample; incubating the fixed bacteria with at least one oligonucleotide as described above and herein, in order to achieve hybridization; removing non-hybridized oligonucleotide; detecting and visualizing pathogenic food-relevant bacterial cells of the species S. aureus with the hybridized oligonucleotide. The methods further can include the step of quantifying the pathogenic food-relevant bacterial cells with the hybridized oligonucleotide. The sample can be a foodstuff sample. The detection can be performed by an optical microscope, an epifluorescence microscope, a chemoluminometer, a fluorometer, or a flow cytometer.

Further embodiments relate to methods for the simultaneous specific detection of bacteria of the genus Campylobacter and the species C. coli and/or C. jejuni in a sample. The methods can include the steps of: cultivating pathogenic food-relevant bacteria contained in a sample; fixing the pathogenic food-relevant bacteria present in the sample; incubating the fixed bacteria with at least one oligonucleotide as described above and herein, in order to achieve hybridization; removing non-hybridized oligonucleotide; detecting and visualizing pathogenic food-relevant bacterial cells of the genus Campylobacter, preferably C. coli or C. jejuni, with the hybridized oligonucleotide. The methods further can include the step of quantifying the pathogenic food-relevant bacterial cells with the hybridized oligonucleotide. The sample can be a foodstuff sample. The detection can be performed by an optical microscope, an epifluorescence microscope, a chemoluminometer, a fluorometer, or a flow cytometer.

Some embodiments relate to kits for performing the methods for the specific detection of bacteria of the genus Listeria, including the species L. monocytogenes; a Staphylococcus of the species S. aureus; or a Campylobacter, including of the species C. coli or C. jejuni; in a sample, as described above and herein. The kits can include at least one oligonucleotide in a hybridization solution, further a washing solution, still further one or more fixation solutions.

### DETAILED DESCRIPTION OF THE PREFERRED EMBODIMENT

In PCR, polymerase chain reaction, a characteristic piece of the respective bacterial genome is amplified with specific primers. If the primer finds its target site, a million-fold amplification of a piece of the inherited material occurs. Upon the following analysis, for example by an agarose gel separating DNA fragments, a qualitative evaluation can take place. In the simplest case this leads to the conclusion that target sites for the primers used were present in the tested sample. Further conclusions are not possible; these target sites can originate from both a living bacterium and a dead bacterium or from naked DNA. Differentiation is not possible with this method. This often leads to false positive results, since the PCR reaction is positive also in the presence of a dead bacterium or naked DNA. A further refinement of this technique is the quantitative PCR, which tries to establish a correlation between the amount of bacteria present and the amount of amplified DNA. Advantages of PCR are its high specificity, its ease of application and its low expenditure of time. Its main disadvantages are its high susceptibility to contamination and therefore false positive results, as well as the aforementioned lack of possibility to discriminate between living and dead cells or naked DNA, respectively.

A unique approach to combine the specificity of molecular biological methods such as PCR with the possibility of the visualization of bacteria, which is facilitated by the antibody methods, is the method of fluorescence in situ hybridization (FISH; RI. Amann, W. Ludwig and K.-H. Schleifer, 1995. “Phylogenetic identification and in situ detection of individual microbial cells without cultivation.” Microbiol. Rev. 59, p. 143-169). Using this method bacteria species, genera or groups can be identified and visualized with high specificity.

The FISH technique is based on the fact that in bacteria cells there are certain molecules which have only been mutated to a small extent in the course of evolution because of their essential function. These are the 16S and the 23S ribosomal ribonucleic acid (rRNA). Both are parts of the ribosomes, the sites of protein biosynthesis, and can serve as specific markers on account of their ubiquitous distribution, their size and their structural and functional constancy (Woese, C. R., 1987. “Bacterial evolution.” Microbiol. Rev. 51, p. 221-271). Based on a comparative sequence analysis, phylogenetic relationships can be established based on these data alone. For this purpose, the sequence data have to be brought into an alignment. In the alignment, which is based on the knowledge about the secondary structure and tertiary structure of these macromolecules, the homologous positions of the ribosomal nucleic acids are brought into line with each other.

Based on these data, phylogenetic calculations can be made. The use of the most modern computer technology makes it possible to make even large-scale calculations fast and effectively, as well as to set up large databases which contain the alignment sequences of the 16S rRNA and 23S rRNA. Because of the fast access to this data material, newly acquired sequences can be phylogenetically analyzed within a short time. These rRNA databases can be used to construct species-specific and genus-specific gene probes. Here all available rRNA sequences are compared with each other and probes are designed for specific sequence sites, which probes cover a specific species, genus or group of bacteria.

In the FISH (fluorescence in situ hybridization) technique, these gene probes, which are complementary to a certain region on the ribosomal target sequence, are brought into the cell. The gene probes are generally small, 16-20 bases long, single-stranded desoxyribonucleic acid pieces and are directed against a target region which is typical for a bacterial species or a bacterial group. If a fluorescence labeled gene probe finds its target sequence in a bacterial cell, it binds to it and the cells can be detected in the fluorescence microscope because of their fluorescence.

The FISH analysis is always performed on a slide, because for the evaluation the bacteria are visualized by irradiation with a high-energy light. But herein lays one of the disadvantages of the classical FISH analysis: because naturally only relatively small volume can be analyzed on the slide, the sensitivity of the method may be unsatisfactory and not sufficient for a reliable analysis. The present invention thus combines the advantages of the classical FISH analysis with those of cultivation. A comparatively short cultivation step ensures that the bacteria to be detected are present in sufficient numbers before the bacteria are detected using specific FISH.

Realization of the methods described in the present application for the simultaneous and specific detection of bacteria of the genus Listeria as well as the species L. monocytogenes or for the specific detection of bacteria of the species S. aureus or for the simultaneous and specific detection of bacteria of the genus Campylobacter as well as the species C. coli and C. jejuni therefore comprises the following steps:
cultivating the bacteria present in the sample to be tested
fixing the bacteria present in the sample
incubating the fixed bacteria with nucleic acid probe molecules, in order to achieve hybridization,
removing or washing off the non-hybridized nucleic acid probe molecules and
detecting the bacteria hybridized with the nucleic acid probe molecules.

Within the scope of the present invention “cultivating” is understood to mean the propagation of the bacteria present in the sample in a suitable cultivation medium. For the detection of Listeria the cultivation may occur, for example, in ½ Fraser bouillon for 24 hours at 30° C. For the detection of S. aureus the cultivation may occur, for example, as blood culture (e.g. BACTEC 9240, Becton Dickinson Instruments) for 8 hours to 48 hours at 35° C. For the detection of Campylobacter the cultivation may occur, for example, in selective medium according to Preston for 24 hours at 42° C. In any case, the expert can find suitable cultivation methods in the prior art.

Within the scope of the present invention “fixing” of the bacteria is understood to mean a treatment with which the bacterial envelope is made permeable for nucleic acid probes. For fixation, usually ethanol is used. If the cell wall cannot be penetrated by the nucleic acid probes using these techniques, the expert will know a sufficient number of other techniques which lead to the same result. These include, for example, methanol, mixtures of alcohols, low percentage paraformaldehyde solution or a diluted formaldehyde solution, enzymatic treatments or the like.

Within the scope of the present invention the fixed bacteria are incubated with fluorescence labeled nucleic acid probes for the “hybridization”. These nucleic acid probes, which consist of an oligonucleotide and a marker linked thereto can then penetrate the cell wall and bind to the target sequence corresponding to the nucleic acid probe in the cell. Binding is to be understood as formation of hydrogen bonds between complementary nucleic acid pieces.

The nucleic acid probe here can be complementary to a chromosomal or episomal DNA, but also to an mRNA or rRNA of the microorganism to be detected. It is advantageous to select a nucleic acid probe which is complementary to a region present in copies of more than 1 in the microorganism to be detected. The sequence to be detected is preferably present in 500-100,000 copies per cell, especially preferred 1,000-50,000 copies. For this reason the rRNA is preferably used as a target site, since the ribosomes as sites of protein biosynthesis are present many thousand fold in each active cell.

The nucleic acid probe within the meaning of the invention may be a DNA or RNA probe comprising usually between 12 and 1,000 nucleotides, preferably between 12 and 500, more preferably between 12 and 200, especially preferably between 12 and 50 and between 15 and 40, and most preferably between 17 and 25 nucleotides. The selection of the nucleic acid probes is done according to criteria of whether a complementary sequence is present in the microorganism to be detected. By selecting a defined sequence, a bacterial species, a bacterial genus or an entire bacterial group may be detected. In a probe consisting of 15 nucleotides, the sequences should be 100% complementary. In oligonucleotides of more than 15 nucleotides, one or more mismatches are allowed.

Within the scope of the methods according to the invention for the simultaneous specific detection of bacteria of the genus Listeria and the species L. monocytogenes the nucleic acid probe molecules of the present invention have the following lengths and sequences:

| | |
|---|---|
| SEQ ID NO. 1: | 5′-GGC TTG CAC CGG CAG TCA CT |
| SEQ ID NO. 2: | 5′-CGG CTT ACA CCG GCA GTC ACT |
| SEQ ID NO. 3: | 5′-CCC TTT GTA CTA TCC ATT GTA |
| SEQ ID NO. 4: | 5′-CCC TTT GTA CCA TCC ATT GTA |
| SEQ ID NO. 5: | 5′-CCC TTT GTA TTA TCC ATT GTA G |
| SEQ ID NO. 6: | 5′-CCC TTT GTA CTG TCC ATT GTA |

For example, the detection method for Listeria and L. monocytogenes is performed as follows: the oligonucleotide SEQ ID NO. 1 is specifically labeled, for example with a green fluorescent dye, and serves for the specific detection of all bacteria of the genus Listeria. The oligonucleotide SEQ ID NO. 2 remains unlabeled and inhibits as competitor the binding of the labeled oligonucleotide SEQ ID NO. 1 to bacteria which do not belong to the genus Listeria. The oligonucleotide of the SEQ ID NO. 3 is also labeled specifically, but differently from the oligonucleotide SEQ ID NO. 1, for example with a red fluorescent dye, and serves for the specific detection of all bacteria of the species Listeria monocytogenes. The oligonucleotides SEQ ID NO. 4, SEQ ID NO. 5 and SEQ ID NO. 6 again remain unlabeled and inhibit as competitors the binding of the labeled oligonucleotide SEQ ID NO. 3 to bacteria which do not belong to the species L. monocytogenes. In this way, the simultaneous and highly specific detection of bacteria belonging to the genus Listeria or to the species L. monocytogenes, respectively, is possible. The different markers, e.g., a green fluorescent dye on the one hand and a red fluorescent dye on the other hand, are easy to distinguish from each other, e.g., by using different filters in the fluorescence microscopy.

Within the scope of the method of the present invention for the specific detection of bacteria of the species S. aureus, the nucleic acid probe molecules of the present invention have the following lengths and sequences:

| | |
|---|---|
| SEQ ID NO. 7: | 5′-GAA GCA AGC TTC TCG TCC G |
| SEQ ID NO. 8: | 5′-GGA GCA AGC TCC TCG TCC G |
| SEQ ID NO. 9: | 5′-GAA GCA AGC TTC TCG TCA TT |
| SEQ ID NO. 10: | 5′-CTA ATG CAG CGC GGA TCC |
| SEQ ID NO. 11: | 5′-CTA ATG CAC CGC GGA TCC |
| SEQ ID NO. 12: | 5′-CTA ATG CGG CGC GGA TCC |
| SEQ ID NO. 13: | 5′-CTA ATG CAG CGC GGG TCC |

For example, the detection method for S. aureus takes place as follows: The oligonucleotides SEQ ID NO. 7 and SEQ ID NO. 10 are labeled specifically, for example with a red fluorescent dye, and serve for the specific detection of all bacteria of the species Staphylococcus aureus. The oligonucleotides SEQ ID NO. 8 and 9 as well as SEQ ID NO. 11, 12 and 13 remain unlabeled and inhibit as competitors the binding of the labeled oligonucleotides to bacteria which do not belong to the species S. aureus. In this way, highly specific detection of bacteria belonging to the species S. aureus is possible.

In a preferred embodiment the intensity of the signals obtained may be enhanced by using so-called “helper probes”. These helper probes are unlabeled oligonucleotides having the following sequence:

| | |
|---|---|
| SEQ ID NO. 14: | TCG CTC GAC TTG CAT GTA TTA GGC A |
| SEQ ID NO. 15: | ACC CGT CCG CCG CTA ACA TCA G |
| SEQ ID NO. 52: | CTA TAA GTG ACA GCA AGA CCG |
| SEQ ID NO. 53: | GTA AGC CGT TAC CTT ACC AAC |

The use of the helper probes is not necessary but optional. The helper probes facilitate the binding of the labeled probes to their target sites and thus enhance the signal intensity. The detection method however also functions very well without these helper probes.

Within the scope of the methods according to the present invention for the simultaneous specific detection of bacteria of the genus Campylobacter and the species C. coli and C. jejuni, the nucleic acid probe molecules of the present invention have the following lengths and sequences:

| | |
|---|---|
| 5′ CTG CCT CTC CCT CAC TCT AG | SEQ ID NO. 16 |
| 5′ CTG CCT CTC CCT TAC TCT AG | SEQ ID NO. 17 |
| 5′ CTG CCT CTC CCC TAC TCT AG | SEQ ID NO. 18 |
| 5′ CTG CCT CTC CCC CAC TCT AG | SEQ ID NO. 19 |
| 5′ CCT ACC TCT CCC ATA CTC TAG A | SEQ ID NO. 20 |
| 5′ CCA TCC TCT CCC ATA CTC TAG C | SEQ ID NO. 21 |
| 5′ CCT ACC TCT CCA GTA CTC TAG T | SEQ ID NO. 22 |
| 5′ CCT GCC TCT CCC ACA CTC TAG A | SEQ ID NO. 23 |
| 5′ CGC TCC GAA AAG TGT CAT CCT C | SEQ ID NO. 24 |
| 5′ CTA AAT ACG TGG GTT GCG | SEQ ID NO. 25 |
| 5′ CTA AAC ACG TGG GTT GCG | SEQ ID NO. 26 |
| 5′ AGC AGA TCG CCT TCG CAA T | SEQ ID NO. 27 |
| 5′ AGC AGA TCG CTT TCG CAA T | SEQ ID NO. 28 |
| 5′ AGT AGA TCG CCT TCG CAA T | SEQ ID NO. 29 |
| 5′ TCG AGT GAA ATC AAC TCC C | SEQ ID NO. 30 |
| 5′ TCG GGT GAA ATC AAC TCC C | SEQ ID NO. 31 |
| 5′ CGT AGC ATG GCT GAT CTA C | SEQ ID NO. 32 |
| 5′ CGT AGC ATA GCT GAT CTA C | SEQ ID NO. 33 |
| 5′ CGT AGC ATT GCT GAT CTA C | SEQ ID NO. 34 |
| 5′ GCC CTG ACT AGC AGA GCA A | SEQ ID NO. 35 |
| 5′ TTC TTG GTG ATC TCT ACG G | SEQ ID NO. 36 |
| 5′ TTC CTG GTG ATC TCT ACG G | SEQ ID NO. 37 |
| 5′ TTC TTG GTG ATA TCT ACG G | SEQ ID NO. 38 |
| 5′ TTG AGT TCT AGC AGA TCG C | SEQ ID NO. 39 |
| 5′ TTG AGT TCC AGC AGA TCG C | SEQ ID NO. 40 |
| 5′ TTG AGT TCT AGC AGA TAG C | SEQ ID NO. 41 |
| 5′ TTG AGT TCC AGC AGA TAG C | SEQ ID NO. 42 |
| 5′ CGC GCC TTA GCG TCA GTT GAG | SEQ ID NO. 43 |
| 5′ CAC GCC TTA GCG TCA GTT GAG | SEQ ID NO. 44 |
| 5′ CGC GCC TTA GCG TCA GTT AAG | SEQ ID NO. 45 |
| 5′ CAC GCA TTA GCG TCA GTT GAG | SEQ ID NO. 46 |
| 5′ CGA GCA TTA GCG TCA GTT GAG | SEQ ID NO. 47 |
| 5′ TAC ACT AGT TGT TGG GGT GG | SEQ ID NO. 48 |
| 5′ TTC GCG CCT CAG CGT CAG TTA CAG | SEQ ID NO. 49 |

The detection method for the genus Campylobacter or the species C. coli and C. jejuni, respectively, is performed as follows: The oligonucleotides SEQ ID NO. 16 to SEQ ID NO. 19 as well as the oligonucleotides SEQ ID NO. 24 to SEQ ID NO. 28 as well as the oligonucleotide SEQ ID NO. 30 as well as the oligonucleotides SEQ ID NO. 32 to 34 are specifically labeled, for example with a green fluorescent dye, and serve for the specific detection of all bacteria of the genus Campylobacter. The oligonucleotides SEQ ID NO. 20 to 23 as well as the oligonucleotide SEQ ID NO. 29 as well as the oligonucleotide SEQ ID NO. 31 remain unlabeled and inhibit as competitors the binding of the aforementioned labeled oligonucleotides which are specific for the genus Campylobacter to bacteria which do not belong to the genus Campylobacter.

The oligonucleotides SEQ ID NO. 35 and 36 as well as the oligonucleotide SEQ ID NO. 39 are also specifically labeled, but differently from the oligonucleotides SEQ ID NO. 16 to 19, 24 to 28, 30 as well as 32 to 34, i.e., distinguishably labeled from them, e.g., with a blue fluorescent dye, and serve for the specific detection of all bacteria of the species Campylobacter coli. The oligonucleotides SEQ ID NO. 37, 38 as well as 40 to 42 again remain unlabeled and inhibit as competitors the binding of the labeled oligonucleotides specific for C. coli to bacteria which do not belong to the species C. coli.

The oligonucleotides SEQ ID NO. 43 and 48 are also specifically labeled, but again differently from the aforementioned oligonucleotides, i.e., again distinguishably labeled from them, e.g., with a red fluorescent dye, and serve for the specific detection of all bacteria of the species Campylobacter jejuni. The oligonucleotides SEQ ID NO. 44 to 47 and 49 again remain unlabeled and inhibit as competitors the binding of the labeled oligonucleotides specific for C. jejuni to bacteria which do not belong to the species C. jejuni.

In this way, the simultaneous and highly specific detection of bacteria belonging to the genus Campylobacter or to the species C. coli or C. jejuni, respectively, is possible.

The intensity of the signals obtained may optionally be enhanced by using so-called helper probes. The helper probes are also unlabeled, but facilitate the binding of the labeled to their target sites and thus enhance the signal intensity. This is just an enhancement of the signal intensity; the detection method of course also functions without these helper probes.

In this way, the intensity of the signals obtained with the oligonucleotide SEQ ID NO. 24 may be enhanced by using the unlabeled oligonucleotides mentioned below as helper probes:

| | |
|---|---|
| SEQ ID NO. 50: | 5′ CAC GCG GCG TTG CTG CTG/T C |
| SEQ ID NO. 51: | 5′ TCT TTT [C/T]CC [A/C/T][G/A]A |
| | [A/C/T]AA AAG GAG TTA CG |

Within the scope of the present invention, competitors are understood to mean in particular oligonucleotides which have a higher specificity for genera or species not to be detected than the labeled oligonucleotides which are specific for the genera or species to be detected.

A further object of the invention are modifications of the above oligonucleotide sequences, demonstrating specific hybridization with target nucleic acid sequences of the respective bacterium despite variations in sequence and/or length, and which are therefore suitable for use in a method according to the invention. These especially include:
a) Nucleic acid molecules (i) being identical to one of the above oligonucleotide sequences (SEQ ID NO. 1 to SEQ ID NO. 53) to at least 60%, 65%, preferably to at least 70%, 75%, more preferably to at least 80%, 84%, 87% and particularly preferred to at least 90%, 94%, 96% of the bases (wherein the sequence region of the nucleic acid molecule is to be considered which corresponds to the sequence region of one of the above oligonucleotides (SEQ ID NO.1 to SEQ ID NO. 53) and not the entire sequence of a nucleic acid molecule, which possibly may be extended by one or multiple bases compared to the above-mentioned oligonucleotides (SEQ ID NO. 1 to SEQ ID NO. 53), or (ii) differs from the above oligonucleotide sequences (SEQ ID NO. 1 to SEQ ID NO. 53) by one or more deletions and/or additions and which render possible a specific hybridization with nucleic acid sequences of bacteria of the genus Listeria and the species L. monocytogenes, of bacteria of the species S. aureus or of bacteria of the genus Campylobacter and the species C. coli and C. jejuni. In this context “specific hybridization” means that under the hybridization conditions described here or those known to the person skilled in the art in relation to in situ hybridization techniques, only the ribosomal RNA of the target organisms binds to the oligonucleotide, but not the rRNA of non-target organisms.
b) Nucleic acid molecules which specifically hybridize to a sequence complementary to the nucleic acid molecules mentioned in a) or to one of the probes SEQ ID NO. 1 to SEQ ID NO. 53 under stringent conditions.
c) Nucleic acid molecules comprising an oligonucleotide sequence of SEQ ID NO. 1 to SEQ ID NO. 53 or the sequence of a nucleic acid molecule according to a) or b) and having at least one further nucleotide in addition to the mentioned sequences or their modifications according to a) or b) and allowing specific hybridization with nucleic acid sequences of target organisms.

The degree of sequence identity of a nucleic acid molecule to the probes SEQ ID NO. 1 to SEQ ID NO. 53 can be determined using the usual algorithms. In this respect, for example, the program for determining the sequence identity available under hypertext transfer protocol (http) available at www.ncbi.nlm.nih.gov/BLAST (on this page for example the link “Standard nucleotide-nucleotide BLAST [blastn]”) is suitable.

In the case of the detection of bacteria of the genus Listeria or the species L. monocytogenes the specific oligonucleotide probes preferably correspond to oligonucleotides SEQ ID NO. 1 or SEQ ID NO. 3. But also modifications are possible, as long as there is still specific hybridization between probe and target sequence. It can be sufficient that the oligonucleotide probe used is identical in 15, preferably 16 and 17 and particularly preferred 18 and 19 successive nucleotides to SEQ ID NO. 1 or SEQ ID NO. 3. The same is true for the oligonucleotides serving as competitors with respect to the sequences SEQ ID NO. 2, 4, 5 and 6.

The same is true for the detection of S. aureus. In this case, the specific oligonucleotide probes preferably have a sequence which is identical to the one of SEQ ID NO. 7 or SEQ ID NO. 10 in 13 and 14 and preferably 15, 16 or 17 successive nucleotides. The same is true for the oligonucleotides serving as competitors with respect to the sequences SEQ ID NO. 8, 9 and 11 to 13.

The same is true for the detection of bacteria of the genus Campylobacter and the species Campylobacter coli and Campylobacter jejuni. Also in this case the specific oligonucleotide probes preferably have a sequence which is identical to SEQ ID NO. 16 to 19, 24 to 28, 30, 32 to 36, 39, 43 and 48 in 13 or 14, preferably 15 or 16 and particularly preferred 17 or 18 successive nucleotides. The same is true for the oligonucleotides serving as competitors with respect to the sequences SEQ ID NO. 20 to 23, 29, 31, 37, 38, 40 to 42, 44 to 47 and 49.

The nucleic acid probe molecules according to the invention may be used within the scope of the detection method with various hybridization solutions. Various organic solvents may be used in concentrations of 0-80%. By keeping stringent hybridization conditions, it is guaranteed that the nucleic acid probe molecule indeed hybridizes to the target sequence. Moderate conditions within the meaning of the invention are e.g. 0% formamide in a hybridization buffer as described below. Stringent conditions within the meaning of the invention are for example 20-80% formamide in the hybridization buffer.

Within the scope of the method according to the invention for simultaneous specific detection of bacteria of the genus Listeria and the species L. monocytogenes a typical hybridization solution contains 0%-80% formamide, preferably 20%-60% formamide, especially preferred 40% formamide. In addition, it has a salt concentration of 0.1 mol/1-1.5 mol/l, preferably of 0.5 mol/l-1.0 mol/l, more preferred of 0.7 mol/l-0.9 mol/l and especially preferred of 0.9 mol/l, the salt preferably being sodium chloride. Further, the hybridization solution usually comprises a detergent, such as for instance sodium dodecyl sulfate (SDS) in a concentration of 0.001%-0.2%, preferably in a concentration of 0.005-0.05%, more preferred of 0.01-0.03%, especially preferred in a concentration of 0.01%. For buffering of the hybridization solution, various compounds such as Tris-HCl, sodium citrate, PIPES or HEPES may be used, which are usually used in concentrations of 0.01-0.1 mol/l, preferably of 0.01 to 0.05 mol/l, in a pH range of 6.0-9.0, preferably 7.0 to 8.0. The particularly preferred inventive embodiment of the hybridization solution contains 0.02 mol/l Tris-HCl, pH 8.0.

Within the scope of the method according to the invention for the specific detection of bacteria of the species S. aureus, a typical hybridization solution contains 0%-80% formamide, preferably 20%-60% formamide, particularly preferred 20% formamide. In addition it has a salt concentration of 0.1 mol/l-1.5 mol/l, preferably of 0.7 mol/l to 0.9 mol/l, particularly preferred of 0.9 mol/l, the salt preferably being sodium chloride. Further, the hybridization solution usually comprises a detergent, such as for example sodium dodecyl sulfate (SDS), in a concentration of 0.001%-0.2%, preferably in a concentration of 0.005-0.05%, more preferably 0.01-0.03%, especially preferred in a concentration of 0.01%. For buffering of the hybridization solution, various compounds such as Tris-HCl, sodium citrate, PIPES or HEPES may be used, which are usually used in concentrations of 0.01-0.1 mol/l, preferably of 0.01 to 0.05 mol/l, in a pH range of 6.0-9.0, preferably 7.0 to 8.0. The particularly preferred inventive embodiment of the hybridization solution contains 0.02 mol/l Tris-HCl, pH 8.0.

Within the scope of the method of the present invention for the specific detection of bacteria of the genus Campylobacter and the species C. coli and C. jejuni, a typical hybridization solution contains 0%-80% formamide, preferably 20%-60% formamide, especially preferred 20% formamide. In addition it has a salt concentration of 0.1 mol/1-1.5 mol/l, preferably of 0.7 mol/l-0.9 mol/l, especially preferred of 0.9 mol/l, the salt preferably being sodium chloride. Further, the hybridization solution usually comprises a detergent such as for example sodium dodecyl sulfate (SDS), in a concentration of 0.001-0.2%, preferably in a concentration of 0.005-0.05%, more preferably 0.01-0.03%, especially preferred in a concentration of 0.01%. For buffering of the hybridization solution, various compounds, such as Tris-HCl, sodium citrate, PIPES or HEPES may be used, which are usually used in concentrations of 0.01-0.1 mol/l, preferably of 0.01 to 0.05 mol/l, in a pH range of 6.0-9.0, preferably 7.0 to 8.0. The particularly preferred inventive embodiment of the hybridization solutions contains 0.02 mol/l Tris-HCl, pH 8.0.

It shall be understood that the expert can choose the given concentrations of the constituents of the hybridization buffer in such a way that the desired stringency of the hybridization reaction is achieved. Especially preferred embodiments reflect stringent to particularly stringent hybridization conditions. Using these stringent conditions the expert can determine whether a particular nucleic acid molecule enables the specific detection of nucleic acid sequences of target organisms, and may thus be reliably used within the scope of the invention. The expert is able to increase or decrease the stringency by variation of the parameters of the hybridization buffer if needed or depending on the probe or the target organism.

The concentration of the nucleic acid probe in the hybridization buffer depends on the kind of label and on the number of target structures. In order to allow rapid and efficient hybridization, the number of nucleic acid probe molecules should exceed the number of target structures by several orders of magnitude. However, it has to be noted that in fluorescence in situ-hybridization (FISH) too high levels of fluorescence labelled nucleic acid probe molecules result in increased background fluorescence. The concentration of the nucleic acid probe molecules should therefore be in the range between 0.5 and 500 ng/μl, preferably between 1.0 and 100 ng/μl, and especially preferred between 1.0-50 ng/μl.

Within the scope of the method of the present invention the preferred concentration is 1-10 ng for each nucleic acid probe molecule used per μl hybridization solution. The volume of the hybridization solution used should be between 8 μl and 100 ml, in an especially preferred embodiment of the method of the present invention it is 30 μl.

The hybridization usually lasts between 10 minutes and 12 hours, preferably the hybridization lasts for about 1.5 hours. The hybridization temperature is preferably between 44° C. and 48° C., especially preferred 46° C., wherein the parameter of the hybridization temperature as well as the concentration of salts and detergents in the hybridization solution may be optimized depending on the nucleic acid probes, especially their lengths and the degree to which they are complementary to the target sequence in the cell to be detected. The expert is familiar with the appropriate calculations.

After hybridization the non-hybridized and excess nucleic acid probe molecules should be removed or washed off, which is usually achieved by a conventional washing solution. This washing solution may, if desired, contain 0.001-0.1%, preferably 0.005-0.05%, especially preferred 0.01%, of a detergent such as SDS, as well as Tris-HCl in a concentration of 0.001-0.1 mol/l, preferably 0.01-0.05 mol/l, especially preferred 0.02 mol/l, wherein the pH value of Tris-HCl is within the range of 6.0 to 9.0, preferably of 7.0 to 8.0, especially preferred 8.0. A detergent may be contained, although this is not absolutely necessary. Furthermore, the washing solution usually contains NaCl, wherein the concentration is 0.003 mol/l to 0.9 mol/l, preferably 0.01 mol/l to 0.9 mol/l, depending on the stringency required. An NaCl concentration of 0.07 mol/l (method for the simultaneous specific detection of bacteria of the genus Listeria and the species L. monocytogenes) or 0.215 mol/l (method for the specific detection of bacteria of the species S. aureus) or 0.215 mol/l (method for the simultaneous specific detection of bacteria of the genus Campylobacter and of the species C. coli and C. jejuni) is especially preferred. Moreover, the washing solution may contain EDTA, wherein the concentration is preferably 0.005 mol/l. The washing solution may further contain suitable amounts of preservatives known to the expert.

Generally, buffer solutions are used in the washing step, which can in principle be very similar to the hybridization buffer (buffered sodium chloride solution), except that the washing step is usually performed in a buffer with a lower salt concentration or at a higher temperature. For theoretical estimation of the hybridization conditions, the following formula may be used:
Td=81.5+16.6 lg[Na⁺]+0.4×(% GC)−820/n−0.5×(% FA)
Td=dissociation temperature in ° C.
[Na⁺]=molarity of the sodium ions
% GC=percentage of guanine and cytosine nucleotides relative to the total number of bases
n=hybrid length
% FA=percentage of formamide

Using this formula, the formamide content (which should be as low as possible due to the toxicity of the formamide) of the washing buffer may for example be replaced by a correspondingly lower sodium chloride content. However, the person skilled in the art knows from the extensive literature concerning in situ hybridization methods the fact that, and in which way, the mentioned contents can be varied. Concerning the stringency of the hybridization conditions, the same applies as outlined above for the hybridization buffer.

The “washing off” of the non-bound nucleic acid probe molecules is usually performed at a temperature in the range of 44° C. to 52° C., preferably of 44° C. to 50° C. and especially preferred at 46° C. for 10 to 40 minutes, preferably for 15 minutes.

In an alternative embodiment of the method according to the invention, the nucleic acid molecules according to the invention are used in the so-called Fast-FISH method for the specific detection of the mentioned target organisms. The Fast-FISH method is known to the expert and is, for example, described in the applications DE 199 36 875 and WO 99/18234. Reference is herewith expressly made to the disclosure contained in these documents regarding the performance of the detection methods described therein.

The specifically hybridized nucleic acid probe molecules can then be detected in the respective cells, provided that the nucleic acid probe molecule is detectable, e.g., by linking the nucleic acid probe molecule to a marker by covalent binding. As detectable markers, for example, fluorescent groups, such as for example CY2 (available from Amersham Life Sciences, Inc., Arlington Heights, USA), CY3 (also available from Amersham Life Sciences), CY5 (also obtainable from Amersham Life Sciences), FITC (Molecular Probes Inc., Eugene, USA), FLUOS (available from Roche Diagnostics GmbH, Mannheim, Germany), TRITC (available from Molecular Probes Inc., Eugene, USA), 6-FAM or FLUOS-PRIME are used, which are well known to the person skilled in the art. Also chemical markers, radioactive markers or enzymatic markers, such as horseradish peroxidase, acid phosphatase, alkaline phosphatase, and peroxidase may be used. For each of these enzymes a number of chromogens are known which may be converted instead of the natural substrate and may be transformed to either coloured or fluorescent products. Examples of such chromogens are listed in the following table:

TABLE

| Enzyme | | Chromogen |
|---|---|---|
| 1. | Alkaline | 4-methylumbelliferyl phosphate (*), bis(4- |
| | phosphatase | methylumbelliferyl phosphate, (*) |
| | and | 3-O-methylfluorescein, flavone-3-diphosphate |
| | acid | triammonium salt (*), p-nitrophenylphosphate |
| | phosphatase | disodium salt |
| 2. | Peroxidase | tyramine hydrochloride (*), 3-(p-hydroxyphenyl)- |
| | | propionate (*), p-hydroxyphenethyl alcohol (*), 2,2′- |
| | | azino-di-3-ethylbenzothiazoline sulfonic acid (ABTS), |
| | | ortho-phenylendiamine dihydrochloride, o-dianisidine, |
| | | 5-aminosalicylic acid, p-ucresol (*), |
| | | 3,3′-dimethyloxy benzidine, 3-methyl-2- |
| | | benzothiazoline hydrazone, tetramethylbenzidine |
| 3. | Horseradish | H₂O₂ + diammonium benzidine |
| | peroxidase | H₂O₂ + tetramethylbenzidine |
| 4. | β-D- | o-nitrophenyl-β-D-galactopyranoside, 4- |
| | galactosidase | methylumbelliferyl-β-D-galactoside |
| 5. | Glucose | ABTS, glucose and thiazolyl blue |
| | oxidase | |

* fluorescence

Finally, it is possible to generate the nucleic acid probe molecules in such a way that another nucleic acid sequence suitable for hybridization is present at their 5′ or 3′ ends. This nucleic acid sequence in turn comprises about 15 to 1,000, preferably 15-50 nucleotides. This second nucleic acid region may in turn be detected by a nucleic acid probe molecule, which is detectable by one of the above-mentioned agents.

Another possibility is the coupling of the detectable nucleic acid probe molecules to a hapten which may subsequently be brought into contact with a hapten-recognizing antibody. Digoxigenin may be mentioned as an example of such a hapten. Other examples in addition to those mentioned are well known to the expert.

The final evaluation depends on the kind of labelling of the probe used and is possible with an optical microscope, epifluorescence microscope, chemoluminometer, fluorometer, etc.

An important advantage of the methods described in this application for the simultaneous specific detection of bacteria of the genus Listeria and the species L. monocytogenes or for the specific detection of bacteria of the species S. aureus, or for the specific detection of bacteria of the genus Campylobacter and the species C. coli and C. jejuni compared to the detection methods described above is the exceptional speed. In comparison to conventional cultivation methods which need up to 10 days, the result is obtained within 24 to 48 hours when the methods according to the invention are used.

Another advantage is the simultaneous detection of bacteria of the genus Listeria and the species L. monocytogenes. With the methods common up to now only bacteria of the species L. monocytogenes are detected more or less reliably. Epidemiological investigations have however shown that besides L. monocytogenes also other species of the genus Listeria can cause the dangerous listeriosis. According to the information presently available, the detection of L. monocytogenes alone is thus not sufficient.

Another advantage is the possibility to discriminate between bacteria of the genus Listeria and those of the species L. monocytogenes. This is easily and reliably possible by using different labels for the nucleic acid probe molecules specific for the corresponding genus or species.

Another advantage is the specificity of these methods. With the nucleic acid probe molecules used, both all species of the genus Listeria, and only the species L. monocytogenes can be specifically detected and visualized. Equally reliably, the species S. aureus and all species of the genus Campylobacter, but also only the species C. coli or C. jejuni are detected with high specificity.

Another advantage is the possibility to discriminate between bacteria of the genus Campylobacter and those of the species C. coli or C. jejuni. This is possible easily and reliably by using different labels for the nucleic acid probe molecules specific for the corresponding genus or species.

By visualization of the bacteria a visual control may be performed at the same time. False-positive results, such as the ones often occurring in polymerase chain reactions, are therefore ruled out.

A further advantage of the methods according to the invention is their ease of use. For example, using these methods, large amounts of samples can easily be tested for the presence of the mentioned bacteria.

The methods according to the invention may be used in various ways.

For example, food samples (e.g., poultry, fresh meat, milk, cheese, vegetables, fruit, fish, etc.) may be tested for the presence of the bacteria to be detected.

For example, also environmental samples may be tested for the presence of bacteria to be detected. These probes may be, for example, collected from soil or be parts of plants.

The method according to the invention may further be used for testing of sewage samples or silage samples.

The method according to the invention may further be used for testing medicinal samples, e.g., stool samples, blood cultures, sputum, tissue samples (also cuts), wound material, urine, samples from the respiratory tract, implants and catheter surfaces.

Another field of application of the method according to the invention is the control of foodstuffs. In preferred embodiments the food samples are obtained from milk or milk products (yogurt, cheese, sweet cheese, butter, and buttermilk), drinking water, beverages (lemonades, beer, and juices), bakery products or meat products.

A further field of application of the method according to the invention is the analysis of pharmaceutical and cosmetic products, e.g. ointments, creams, tinctures, juices, solutions, drops, etc.

Furthermore, according to the invention, kits for performing the respective methods are provided. The hybridization arrangement contained in these kits is described for example in German patent application 100 61 655.0. Express reference is herewith made to the disclosure contained in this document with respect to the in situ hybridization arrangement.

Besides the described hybridization arrangement (referred to as VIT reactor), the most important component of the kits is the respective hybridization solution (referred to as VIT solution) with the nucleic acid probe molecules specific for the microorganisms to be detected, which are described above (VIT solution). Further contained are the respective hybridization buffer (Solution C) and a concentrate of the respective washing solution (Solution D). Also contained are optionally fixation solutions (Solution A (50% ethanol) and Solution B (absolute ethanol)) as well as optionally an embedding solution (finisher). Finishers are commercially available; they prevent, among other things, the rapid bleaching of fluorescent probes under the fluorescence microscope. Optionally, solutions for parallel carrying out of a positive control as well as of a negative control are contained.

The following example is intended to illustrate the invention without limiting it. The buffers and solutions used have the compositions given above.

### EXAMPLE

### Specific Rapid Detection of Pathogenic Food-Relevant Bacteria in a Sample

A sample is cultivated for 20 to 44 hours in a suitable manner. For the detection of Listeria cultivation may be performed for example in ½ Fraser bouillon for 24 hours at 30° C. For the detection of S. aureus the cultivation may be performed for example as blood culture (e.g. BACTEC 9240, Becton Dickinson Instruments) for 8 hours to 48 hours at 35° C. For the detection of Campylobacter the cultivation may be performed, for example, in selective medium according to Preston for 24 hours at 42° C.

To an aliquot of the culture the same volume of fixation solution (Solution B) is added. Alternatively, an aliquot of the culture may be centrifuged (4000 g, 5 min, room temperature) and, after discarding the supernatant, the pellet may be dissolved in 4 drops of fixation solution.

For performing the hybridization a suitable aliquot of the fixed cells (preferably 40 μl) is applied onto a slide and dried (46° C., 30 min, or until completely dry). Alternatively, the cells may also be applied to other carrier materials (e.g. a microtiter plate or a filter). The dried cells are then completely dehydrated by again adding the fixation solution (Solution B, preferably 40 μl). The slide is again dried (room temperature, 3 min, or until completely dry).

Then the hybridization solution (VIT solution) containing the above described nucleic acid probe molecules specific for the microorganisms to be detected is applied to the fixed, dehydrated cells. The preferred volume is 40 μl. The slide is then incubated in a chamber humidified with hybridization buffer (Solution C, corresponding to the hybridization solution without probe molecules), preferably the VIT reactor (46° C., 90 min).

Then the slide is removed from the chamber, the chamber is filled with washing solution (Solution D, diluted 1:10 with distilled water) and the slide is incubated in the chamber (46° C., 15 min).

Then the chamber is filled with distilled water, the slide is briefly immersed and then air-dried in lateral position (46° C., 30 min or until completely dry).

Then the slide is embedded in a suitable medium (finisher).

Finally, the sample is analyzed with the help of a fluorescence microscope.

1. A method for the simultaneous specific detection of bacteria of the genus Listeria and the species L. monocytogenes in a sample, comprising the steps:
a) cultivating the pathogenic food-relevant bacteria contained in the sample;
b) fixing the pathogenic food-relevant bacteria present in the sample;
c) incubating the fixed bacteria with at least one oligonucleotide selected from the group consisting of:
i)
SEQ ID No. 1: 5′-ggc ttg cac cgg cag tca ct,
SEQ ID No. 2: 5′-cgg ctt aca ccg gca gtc act,
SEQ ID No. 3: 5′-ccc ttt gta cta tcc att gta,
SEQ ID No. 4: 5′-ccc ttt gta cca tcc att gta,
SEQ ID No. 5: 5′-ccc ttt gta tta tcc att gta g, and
SEQ ID No. 6: 5′-ccc ttt gta ctg tcc att gta,
ii) an oligonucleotide which has at least 60% of the bases identical to an oligonucleotide according to i) and which renders possible a specific hybridization with nucleic acid sequences of the bacteria of the genus Listeria and/or the species L. monocytogenes,
(iii) an oligonucleotide which differs from the oligonucleotide according to i) and ii) in that it is extended by at least one nucleotide, and
iv) an oligonucleotide which hybridizes with a sequence complementary to an oligonucleotide according to i), ii) or iii) under stringent conditions, in order to achieve hybridization,
d) removing non-hybridized oligonucleotides; and
e) detecting and visualizing the pathogenic food-relevant bacterial cells of the genus Listeria and/or the species Listeria monocytogenes with the hybridized oligonucleotide.

2. The method of claim 1, further comprising quantifying the pathogenic food-relevant bacterial cells with the hybridized oligonucleotide.

3. The method according to claim 1, wherein the sample is a foodstuff sample.

4. The method according to claim 1, wherein the detection is performed by an optical microscope, epifluorescence microscope, chemoluminometer, fluorometer, or flow cytometer.

5. A method for the specific detection of bacteria of the species S. aureus in a sample, comprising the steps:
a) cultivating the pathogenic food-relevant bacteria contained in the sample;
b) fixing the pathogenic food-relevant bacteria present in the sample;
c) incubating the fixed bacteria with at least one oligonucleotide selected from the group consisting of:
i)
SEQ ID No. 7: 5′-GAA GCA AGC TTC TCG TCC G,
SEQ ID No. 8: 5′-GGA GCA AGC TCC TCG TCC G,
SEQ ID No. 9: 5′-GAA GCA AGC TTC TCG TCA TT,
SEQ ID No. 10: 5′-CTA ATG CAG CGC GGA TCC,
SEQ ID No. 11: 5′-CTA ATG CAC CGC GGA TCC,
SEQ ID No. 12: 5′-CTA ATG CGG CGC GGA TCC, and
SEQ ID No. 13: 5′-CTA ATG CAG CGC GGG TCC,
ii) an oligonucleotide which has at least 60% bases identical to an oligonucleotide according to i) and which renders possible a specific hybridization with a nucleic acid sequence of the species S. aureus,
iii) an oligonucleotide which differs from the oligonucleotide according to i) and ii) in that it is extended by at least one nucleotide, and
iv) an oligonucleotide which hybridizes with a sequence complementary to an oligonucleotide according to i), ii) or iii) under stringent conditions in order to achieve hybridization,
d) removing non-hybridized oligonucleotides; and
e) detecting and visualizing the pathogenic food-relevant bacterial cells of the species Staphylococcus aureus with the hybridized oligonucleotides.

6. The method of claim 5, further comprising quantifying the pathogenic food-relevant bacterial cells with the hybridized oligonucleotide.

7. The method according to claim 5, wherein the sample is a foodstuff sample.

8. The method according to claim 5, wherein the detection is performed by an optical microscope, epifluorescence microscope, chemoluminometer, fluorometer, or flow cytometer.

9. A method for the simultaneous specific detection of bacteria of the genus Campylobacter and the species C. coli and/or C. jejuni in a sample, comprising the steps:
a) cultivating the pathogenic food-relevant bacteria contained in the sample;
b) fixing the pathogenic food-relevant bacteria present in the sample;
c) incubating the fixed bacteria with at least one oligonucleotide selected from the group consisting of:
i)
SEQ ID NO. 16 5′ CTG CCT CTC CCT CAC TCT AG,
SEQ ID NO. 17 5′ CTG CCT CTC CCT TAC TCT AG,
SEQ ID NO. 18 5′ CTG CCT CTC CCC TAC TCT AG,
SEQ ID NO. 19 5′ CTG CCT CTC CCC CAC TCT AG,
SEQ ID NO. 20 5′ CCT ACC TCT CCC ATA CTC TAG A,
SEQ ID NO. 21 5′ CCA TCC TCT CCC ATA CTC TAG C,
SEQ ID NO. 22 5′ CCT ACC TCT CCA GTA CTC TAG T,
SEQ ID NO. 23 5′ CCT GCC TCT CCC ACA CTC TAG A,
SEQ ID NO. 24 5′ CGC TCC GAA AAG TGT CAT CCT C,
SEQ ID NO. 25 5′ CTA AAT ACG TGG GTT GCG,
SEQ ID NO. 26 5′ CTA AAC ACG TGG GTT GCG,
SEQ ID NO. 27 5′ AGC AGA TCG CCT TCG CAA T,
SEQ ID NO. 28 5′ AGC AGA TCG CTT TCG CAA T,
SEQ ID NO. 29 5′ AGT AGA TCG CCT TCG CAA T,
SEQ ID NO. 30 5′ TCG AGT GAA ATC AAC TCC C,
SEQ ID NO. 31 5′ TCG GGT GAA ATC AAC TCC C,
SEQ ID NO. 32 5′ CGT AGC ATG GCT GAT CTA C,
SEQ ID NO. 33 5′ CGT AGC ATA GCT GAT CTA C,
SEQ ID NO. 34 5′ CGT AGC ATT GCT GAT CTA C,
SEQ ID NO. 35 5′ GCC CTG ACT AGC AGA GCA A,
SEQ ID NO. 36 5′ TTC TTG GTG ATC TCT ACG G,
SEQ ID NO. 37 5′ TTC CTG GTG ATC TCT ACG G,
SEQ ID NO. 38 5′ TTC TTG GTG ATA TCT ACG G,
SEQ ID NO. 39 5′ TTG AGT TCT AGC AGA TCG C,
SEQ ID NO. 40 5′ TTG AGT TCC AGC AGA TCG C,
SEQ ID NO. 41 5′ TTG AGT TCT AGC AGA TAG C,
SEQ ID NO. 42 5′ TTG AGT TCC AGC AGA TAG C,
SEQ ID NO. 43 5′ CGC GCC TTA GCG TCA GTT GAG,
SEQ ID NO. 44 5′ CAC GCC TTA GCG TCA GTT GAG,
SEQ ID NO. 45 5′ CGC GCC TTA GCG TCA GTT AAG,
SEQ ID NO. 46 5′ CAC GCA TTA GCG TCA GTT GAG,
SEQ ID NO. 47 5′ CGA GCA TTA GCG TCA GTT GAG,
SEQ ID NO. 48 5′ TAC ACT AGT TGT TGG GGT GG, and
SEQ ID NO. 49 5′ TTC GCG CCT CAG CGT CAG TTA CAG,
ii) an oligonucleotide which has at least 60% of the bases identical to one of the oligonucleotides according to i) and which renders possible a specific hybridization with a nucleic acid sequences of bacteria of the genus Campylobacter and/or the species C. coli and/or C. jejuni,
iii) an oligonucleotide which differs from the oligonucleotide according to i) and ii) in that it is extended by at least one nucleotide, and
iv) an oligonucleotide which hybridize with a sequence complementary to an oligonucleotide according to i), ii) or iii) under stringent conditions in order to achieve hybridization,
d) removing non-hybridized oligonucleotide; and
e) detecting and visualizing the pathogenic food-relevant bacterial cells of the genus Campylobacter and/or the species C. coli and/or C. jejuni with the hybridized oligonucleotide.

10. The method of claim 9, further comprising quantifying the pathogenic food-relevant bacterial cells with the hybridized oligonucleotide.

11. The method according to claim 9, wherein the sample is a foodstuff sample.

12. The method according to claim 9, wherein the detection is performed by an optical microscope, epifluorescence microscope, chemoluminometer, fluorometer, or flow cytometer.

13. A kit for performing the method according to claim 1.

14. The kit according to claim 13, comprising at least one oligonucleotide in a hybridization solution.

15. The kit according to claim 13, comprising a washing solution.

16. The kit according to claim 13, comprising one or more fixation solutions.

17. A kit for performing the method according to claim 5.

18. The kit according to claim 17, comprising at least one oligonucleotide in a hybridization solution.

19. The kit according to claim 17, comprising a washing solution.

20. The kit according to claim 17, comprising one or more fixation solutions.

21. A kit for performing the method according to claim 9.

22. The kit according to claim 21, comprising at least one oligonucleotide in a hybridization solution.

23. The kit according to claim 21, comprising a washing solution.

24. The kit according to claim 21, comprising one or more fixation solutions.


SEQUENCE LISTING (53 sequences)

SEQ ID NO: 1 - DNA, 20 bp, Artificial Sequence
  oligonucleotide probe
ggcttgcaccggcagtcact

SEQ ID NO: 2 - DNA, 21 bp, Artificial Sequence
  oligonucleotide probe
cggcttacaccggcagtcact

SEQ ID NO: 3 - DNA, 21 bp, Artificial Sequence
  oligonucleotide probe
ccctttgtactatccattgta

SEQ ID NO: 4 - DNA, 21 bp, Artificial Sequence
  oligonucleotide probe
ccctttgtaccatccattgta

SEQ ID NO: 5 - DNA, 22 bp, Artificial Sequence
  oligonucleotide probe
ccctttgtattatccattgtag

SEQ ID NO: 6 - DNA, 21 bp, Artificial Sequence
  oligonucleotide probe
ccctttgtactgtccattgta

SEQ ID NO: 7 - DNA, 19 bp, Artificial Sequence
  oligonucleotide probe
gaagcaagcttctcgtccg

SEQ ID NO: 8 - DNA, 19 bp, Artificial Sequence
  oligonucleotide probe
ggagcaagctcctcgtccg

SEQ ID NO: 9 - DNA, 20 bp, Artificial Sequence
  oligonucleotide probe
gaagcaagcttctcgtcatt

SEQ ID NO: 10 - DNA, 18 bp, Artificial Sequence
  oligonucleotide probe
ctaatgcagcgcggatcc

SEQ ID NO: 11 - DNA, 18 bp, Artificial Sequence
  oligonucleotide probe
ctaatgcaccgcggatcc

SEQ ID NO: 12 - DNA, 18 bp, Artificial Sequence
  oligonucleotide probe
ctaatgcggcgcggatcc

SEQ ID NO: 13 - DNA, 18 bp, Artificial Sequence
  oligonucleotide probe
ctaatgcagcgcgggtcc

SEQ ID NO: 14 - DNA, 25 bp, Artificial Sequence
  oligonucleotide probe
tcgctcgacttgcatgtattaggca

SEQ ID NO: 15 - DNA, 22 bp, Artificial Sequence
  oligonucleotide probe
acccgtccgccgctaacatcag

SEQ ID NO: 16 - DNA, 20 bp, Artificial Sequence
  oligonucleotide probe
ctgcctctccctcactctag

SEQ ID NO: 17 - DNA, 20 bp, Artificial Sequence
  oligonucleotide probe
ctgcctctcccttactctag

SEQ ID NO: 18 - DNA, 20 bp, Artificial Sequence
  oligonucleotide probe
ctgcctctcccctactctag

SEQ ID NO: 19 - DNA, 20 bp, Artificial Sequence
  oligonucleotide probe
ctgcctctcccccactctag

SEQ ID NO: 20 - DNA, 22 bp, Artificial Sequence
  oligonucleotide probe
cctacctctcccatactctaga

SEQ ID NO: 21 - DNA, 22 bp, Artificial Sequence
  oligonucleotide probe
ccatcctctcccatactctagc

SEQ ID NO: 22 - DNA, 22 bp, Artificial Sequence
  oligonucleotide probe
cctacctctccagtactctagt

SEQ ID NO: 23 - DNA, 22 bp, Artificial Sequence
  oligonucleotide probe
cctgcctctcccacactctaga

SEQ ID NO: 24 - DNA, 22 bp, Artificial Sequence
  oligonucleotide probe
cgctccgaaaagtgtcatcctc

SEQ ID NO: 25 - DNA, 18 bp, Artificial Sequence
  oligonucleotide probe
ctaaatacgtgggttgcg

SEQ ID NO: 26 - DNA, 18 bp, Artificial Sequence
  oligonucleotide probe
ctaaacacgtgggttgcg

SEQ ID NO: 27 - DNA, 19 bp, Artificial Sequence
  oligonucleotide probe
agcagatcgccttcgcaat

SEQ ID NO: 28 - DNA, 19 bp, Artificial Sequence
  oligonucleotide probe
agcagatcgctttcgcaat

SEQ ID NO: 29 - DNA, 19 bp, Artificial Sequence
  oligonucleotide probe
agtagatcgccttcgcaat

SEQ ID NO: 30 - DNA, 19 bp, Artificial Sequence
  oligonucleotide probe
tcgagtgaaatcaactccc

SEQ ID NO: 31 - DNA, 19 bp, Artificial Sequence
  oligonucleotide probe
tcgggtgaaatcaactccc

SEQ ID NO: 32 - DNA, 19 bp, Artificial Sequence
  oligonucleotide probe
cgtagcatggctgatctac

SEQ ID NO: 33 - DNA, 19 bp, Artificial Sequence
  oligonucleotide probe
cgtagcatagctgatctac

SEQ ID NO: 34 - DNA, 19 bp, Artificial Sequence
  oligonucleotide probe
cgtagcattgctgatctac

SEQ ID NO: 35 - DNA, 19 bp, Artificial Sequence
  oligonucleotide probe
gccctgactagcagagcaa

SEQ ID NO: 36 - DNA, 19 bp, Artificial Sequence
  oligonucleotide probe
ttcttggtgatctctacgg

SEQ ID NO: 37 - DNA, 19 bp, Artificial Sequence
  oligonucleotide probe
ttcctggtgatctctacgg

SEQ ID NO: 38 - DNA, 19 bp, Artificial Sequence
  oligonucleotide probe
ttcttggtgatatctacgg

SEQ ID NO: 39 - DNA, 19 bp, Artificial Sequence
  oligonucleotide probe
ttgagttctagcagatcgc

SEQ ID NO: 40 - DNA, 19 bp, Artificial Sequence
  oligonucleotide probe
ttgagttccagcagatcgc

SEQ ID NO: 41 - DNA, 19 bp, Artificial Sequence
  oligonucleotide probe
ttgagttctagcagatagc

SEQ ID NO: 42 - DNA, 19 bp, Artificial Sequence
  oligonucleotide probe
ttgagttccagcagatagc

SEQ ID NO: 43 - DNA, 21 bp, Artificial Sequence
  oligonucleotide probe
cgcgccttagcgtcagttgag

SEQ ID NO: 44 - DNA, 21 bp, Artificial Sequence
  oligonucleotide probe
cacgccttagcgtcagttgag

SEQ ID NO: 45 - DNA, 21 bp, Artificial Sequence
  oligonucleotide probe
cgcgccttagcgtcagttaag

SEQ ID NO: 46 - DNA, 21 bp, Artificial Sequence
  oligonucleotide probe
cacgcattagcgtcagttgag

SEQ ID NO: 47 - DNA, 21 bp, Artificial Sequence
  oligonucleotide probe
cgagcattagcgtcagttgag

SEQ ID NO: 48 - DNA, 20 bp, Artificial Sequence
  oligonucleotide probe
tacactagttgttggggtgg

SEQ ID NO: 49 - DNA, 24 bp, Artificial Sequence
  oligonucleotide probe
ttcgcgcctcagcgtcagttacag

SEQ ID NO: 50 - DNA, 19 bp, Artificial Sequence
  oligonucleotide probe
cacgcggcgttgctgctkc

SEQ ID NO: 51 - DNA, 26 bp, Artificial Sequence
  oligonucleotide probe
tcttttycchrahaaaaggagttacg

SEQ ID NO: 52 - DNA, 21 bp, Artificial Sequence
  oligonucleotide probe
ctataagtgacagcaagaccg

SEQ ID NO: 53 - DNA, 21 bp, Artificial Sequence
  oligonucleotide probe
gtaagccgttaccttaccaac